(19) United States
(12) Patent Application Publication
(10) Pub. No.: US 2024/0423885 A1
(43) Pub. Date: Dec. 26, 2024

(54) HAIR DYE COMPOSITION

(71) Applicant: DARIYA CO., LTD., Nagoya-shi (JP)

(72) Inventor: Kenji IRIAI, Inazawa-shi (JP)

(21) Appl. No.: 18/827,004

(22) Filed: Sep. 6, 2024

Related U.S. Application Data
(63) Continuation of application No. PCT/JP2023/002051, filed on Jan. 24, 2023.

(51) Int. Cl. A61K 8/34; A61K 8/31; A61Q 5/10

(52) U.S. Cl. CPC A61K 8/342; A61K 8/31; A61Q 5/10

(57) ABSTRACT

A hair dye composition, in which a first agent and a second agent have good preservation stability and good mixability, is provided. The hair dye composition includes the first agent containing an alkaline agent and an oxidative dye, and the second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C.

### CROSS-REFERENCE TO RELATED APPLICATION

This application is a continuation application of PCT Application No. PCT/JP2023/002051, filed on Jan. 24, 2023, which is incorporated by reference for all purposes.

### BACKGROUND

The present invention relates to a hair dye composition, and particularly relates to a hair dye composition, in which a first agent and a second agent have good preservation stability and good mixability.

With the increase in fashion consciousness in recent years, oxidative hair dyes (hereafter referred to as hair dyes) have become widely used as a method to change the appearance of hair. A hair dye exhibits excellent color development and color tone persistence through the use of the mixture of a first agent containing an alkali agent and an oxidative dye and a second agent containing an oxidizing agent, which are mixed upon use. Therefore, hair dyes containing large amounts of alkaline agents, oxidative dyes, and oxidizing agents are known to improve the color development of the finish. Furthermore, it is known that adjusting the balance of the types of oxidative dyes improves the color retention after hair dyeing. An example of such a hair dye that improves color retention after hair dyeing is the one in Japanese Patent Laid-Open No. 2009-29754.

In Japanese Patent Laid-Open No. 2009-29754, changes in hair dye color tone have been examined over a short storage period, but preservation stability with respect to the appearance, such as the formation of water droplets or separation after long-term storage, has not been examined. Also, depending on the ingredients of a preparation, mixability of the first agent and the second agent may deteriorate, and the mixability has not been examined.

Accordingly, an objective of the present invention is to provide a hair dye composition, in which a first agent and a second agent have good preservation stability and good mixability.

### SUMMARY

As a result of intensive research into solving the above problems, the present inventors have discovered that a hair dye composition including a first agent containing an alkaline agent and an oxidative dye and a second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C., and have good preservation stability and good mixability. Hence, the present inventors have completed the present invention.

The present invention provides a hair dye composition, in which a first agent and a second agent have good preservation stability and good mixability.

### DETAILED DESCRIPTION

The present invention is described in detail below. All units indicating contents are mass % unless otherwise specified.

The present invention relates to a hair dye composition including a first agent containing an alkaline agent and an oxidative dye and a second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C.

The first agent of the hair dye composition in the present invention will be described.

The first agent of the hair dye composition of the present invention contains nonionic surfactant (A) from the viewpoint of the preservation stability of the first agent and the second agent and the mixability of the first agent and the second agent.

Examples of ingredient (A) above used in the first agent of the present invention include, but are not particularly limited to, polyoxyethylene (hereinafter referred to as POE) alkyl ethers, POE polyoxypropylene (hereinafter referred to as POP) alkyl ethers, POE fatty acid esters, POE sorbitan fatty acid esters, POE castor oil, POE hardened castor oil, POE sorbitol tetra fatty acid esters, POE phytosterols, glycerol fatty acid esters, sorbitan fatty acid esters, polyglycerol fatty acid esters, sucrose fatty acid esters, alkyl polyglucosides, N-alkyl dimethyl amine oxides, and alkanolamides, and one or two or more types thereof may be contained.

Among the examples of ingredient (A) above used in the first agent of the present invention, POE alkyl ethers are preferred from the viewpoint of preservation stability.

Examples of the POE alkyl ethers as ingredient (A) above used in the first agent of the present invention include, but are not particularly limited to, POE (2) lauryl ether, POE (4.2) lauryl ether, POE (9) lauryl ether, POE (21) lauryl ether, POE (25) lauryl ether, POE (2) cetyl ether, POE (5) cetyl ether, POE (5.5) cetyl ether, POE (7) cetyl ether, POE (8) cetyl ether, POE (10) cetyl ether, POE (13) cetyl ether, POE (15) cetyl ether, POE (20) cetyl ether, POE (23) cetyl ether, POE (25) cetyl ether, POE (30) cetyl ether, POE (40) cetyl ether, POE (2) stearyl ether, POE (4) stearyl ether, POE (11) stearyl ether, POE (15) stearyl ether, POE (20) stearyl ether, POE (30) stearyl ether, POE (40) stearyl ether, POE (2) oleyl ether, POE (7) oleyl ether, POE (10) oleyl ether, POE (15) oleyl ether, POE (20) oleyl ether, POE (50) oleyl ether, POE (5) behenyl ether, POE (10) behenyl ether, POE (20) behenyl ether, and POE (30) behenyl ether, and one or two or more types thereof may be contained. Of these, POE (13) cetyl ether, POE (15) cetyl ether, and POE (40) cetyl ether are preferred from the viewpoint of improving preservation stability. The numerals in parentheses following POE each refer to the number of moles of ethylene oxide added.

From the viewpoint of improving preservation stability, two or more types of ingredient (A) above used in the first agent of the present invention are preferably contained.

The content of ingredient (A) above used in the first agent of the present invention is not particularly limited, but ranges from preferably 1.2% to 5.4%, and more preferably 2.1% to 4.2% from the viewpoint of the preservation stability of the first agent and the mixability of the first agent and the second agent. If the content of ingredient (A) above is less than 1.2%, the preservation stability of the first agent and the mixability of the first agent and the second agent may deteriorate. If the content of ingredient (A) above exceeds 5.4%, the mixability of the first agent and the second agent may deteriorate.

The first agent of the hair dye composition in the present invention contains higher alcohol (B) having 12 to 24 carbon atoms from the viewpoint of the preservation stability of the first agent and the mixability of the first agent and the second agent.

Examples of ingredient (B) above used in the first agent of the present invention include, but are not particularly limited to, lauryl alcohol, myristyl alcohol, cetanol, stearyl alcohol, isostearyl alcohol, oleyl alcohol, arachyl alcohol, behenyl alcohol, octyldodecanol, and decyltetradecanol, and one or two more thereof may be contained. Of these, cetanol and stearyl alcohol are preferred from the viewpoint of improving preservation stability.

From the viewpoint of improving preservation stability, two or more types of ingredient (B) above used in the first agent of the present invention are preferably contained.

The content of ingredient (B) above used in the first agent of the present invention is not particularly limited, but ranges from preferably 2.7% to 7.2%, and more preferably 3.6% to 6.3% from the viewpoint of the mixability of the first agent and the second agent. If the content of ingredient (B) above is less than 2.7% or more than 7.2%, the mixability of the first agent and the second agent may deteriorate.

The first agent of the hair dye composition of the present invention contains hydrocarbon (C) that is in a liquid or paste form at 25° C. from the viewpoint of the preservation stability of the first agent and the mixability of the first agent and the second agent.

Regarding ingredient (C) above used in the first agent of the present invention, examples of the hydrocarbon that is in a liquid form at 25° C. include, but are not particularly limited to, paraffin, liquid paraffin, liquid isoparaffin, light liquid isoparaffin, heavy liquid isoparaffin, squalane, and vegetable squalane, and examples of the hydrocarbon that is in a paste form at 25° C. include, but are not particularly limited to, vaseline, and one or two or more types thereof may be contained. Of these, liquid paraffin, liquid isoparaffin, and squalane are preferred from the viewpoint of improving the preservation stability of the first agent, and liquid paraffin is more preferred from the viewpoint of improving the mixability of the first agent and the second agent.

The content of ingredient (C) above used in the first agent of the present invention is not particularly limited, but preferably ranges from 0.1% to 10%, and more preferably 1% to 8% from the viewpoint of the preservation stability of the first agent and the mixability of the first agent and the second agent. If the content of ingredient (C) above is less than 0.1%, the mixability of the first agent and the second agent may deteriorate. If the content of ingredient (C) above exceeds 10%, the preservation stability of the first agent and the mixability of the first agent and the second agent may deteriorate.

The first agent of the hair dye composition of the present invention contains an alkaline agent from the viewpoint of hair dyeability.

Examples of the alkaline agent used in the first agent of the present invention include, but are not particularly limited to, ammonia, monoethanolamine, ammonium hydrogencarbonate, sodium hydrogencarbonate, and sodium carbonate, and one or two or more types thereof may be contained.

The content of the alkaline agent used in the first agent of the present invention is not particularly limited, but preferably ranges from 0.1% to 10%.

The first agent of the hair dye composition in the present invention may contain an oxidative dye from the viewpoint of hair dyeability.

Examples of the oxidative dye used in the first agent of the present invention include, but are not particularly limited to, 2,4-diaminophenoxyethanol, toluene-2,5-diamine, para-aminophenol, paraphenylenediamine, meta-aminophenol, metaphenylenediamine, resorcin, 5-amino-orthocresol and salts thereof, and one or two or more types thereof may be contained. Specific examples of salts of these oxidative dyes include hydrochlorides and sulfates.

The content of the oxidative dye used in the first agent of the present invention is not particularly limited, but preferably ranges from 0.01% to 10%.

The viscosity of the first agent of the present invention at 20° C. is not particularly limited, but preferably ranges from 100 mPa·s to 100,000 mPa·s from the viewpoint of the mixability of the first agent and the second agent.

The viscosity of the first agent of the present invention at 20° C. was measured by filling a sample bottle (Food 140: Manufactured by Dai-ichi Glass Co., Ltd.) with 120 g of the first agent prepared by a usual method and allowing it to stand at 20° C. for 24 hours, and then rotating it for 1 minute: at 60 rpm using an M4 rotor when the viscosity was 100 mPa·s or more and less than 10,000 mPa·s as measured with a B-type viscometer (Model: Digital Viscometer TVB-10M, manufactured by Toki Sangyo Co., Ltd); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; or at 6 rpm using the M4 rotor when the viscosity was 50,000 mPa·s or more and 100,000 mPa·s or less as measured with the same.

The pH of the first agent of the present invention at 20° C. is not particularly limited, but preferably ranges from pH 9 to 12.

The pH of the first agent of the present invention at 20° C. is measured by filling a sample bottle (Food 140: Manufactured by Daiichi Glass Co., Ltd.) with 120 g of the first agent prepared by the usual method, allowing it to stand at 20° C. for 24 hours, and then measuring the pH of the stock solution using a glass electrode hydrogen ion concentration indicator (F-71, manufactured by HORIBA, Ltd.).

Examples of the formulation of the first agent of the present invention include, but are not particularly limited to, cream, emulsion and liquid. Of these, cream or emulsion is preferred from the viewpoint of usability.

Examples of the container to be filled with the first agent of the present invention include, but are not particularly limited to, tubes, pouch films, and aerosols.

The second agent of the hair dye composition in the present invention will be described.

The second agent of the hair dye composition of the present invention contains nonionic surfactant (A) from the viewpoint of the preservation stability of the second agent and the mixability of the first agent and the second agent.

Examples of ingredient (A) above used in the second agent of the present invention include, but are not particularly limited to, POE alkyl ethers, POEPOP alkyl ethers, POE fatty acid esters, POE sorbitan fatty acid esters, POE castor oil, POE hardened castor oil, POE sorbitol tetra fatty acid esters, POE phytosterols, glycerol fatty acid esters, sorbitan fatty acid esters, polyglycerol fatty acid esters, sucrose fatty acid esters, alkyl polyglucosides, N-alkyldimethylamine oxides, and alkanolamides, and one or two or more types thereof may be contained.

Among the examples of ingredient (A) above used in the second agent of the present invention, POE alkyl ethers are preferred from the viewpoint of preservation stability.

Examples of the POE alkyl ether as ingredient (A) used in the second agent of the present invention include, but are not particularly limited to, POE (2) lauryl ether, POE (4.2) lauryl ether, POE (9) lauryl ether, POE (21) lauryl ether, POE (25) lauryl ether, POE (2) cetyl ether, POE (5) cetyl ether, POE (5.5) cetyl ether, POE (7) cetyl ether, POE (8) cetyl ether, POE (10) cetyl ether, POE (13) cetyl ether, POE (15) cetyl ether, POE (20) cetyl ether, POE (23) cetyl ether, POE (25) cetyl ether, POE (30) cetyl ether, POE (40) cetyl ether, POE (2) stearyl ether, POE (4) stearyl ether, POE (11) stearyl ether, POE (15) stearyl ether, POE (20) stearyl ether, POE (30) stearyl ether, POE (40) stearyl ether, POE (2) oleyl ether, POE (7) oleyl ether, POE (10) oleyl ether, POE (15) oleyl ether, POE (20) oleyl ether, POE (50) oleyl ether, POE (5) behenyl ether, POE (10) behenyl ether, POE (20) behenyl ether, and POE (30) behenyl ether, and one or two or more types thereof may be contained. Of these, POE (10) cetyl ether, POE (20) cetyl ether, POE (20) stearyl ether, and POE (30) stearyl ether are preferred from the viewpoint of improving preservation stability.

Two or more types of ingredient (A) above used in the second agent of the present invention are preferably contained from the viewpoint of improving preservation stability.

The content of ingredient (A) above used in the second agent of the present invention is not particularly limited, but ranges from preferably 0.26% to 4.2%, and more preferably 1.3% to 3.4% from the viewpoint of the preservation stability of the second agent and the mixability of the first agent and the second agent. If the content of ingredient (A) above is less than 0.26%, the preservation stability of the second agent and the mixability of the first agent and the second agent may deteriorate. If the content of ingredient (A) above exceeds 4.2%, the mixability of the first agent and the second agent may deteriorate.

The second agent of the hair dye composition in the present invention contains higher alcohol (B) having 12 to 24 carbon atoms from the viewpoint of the preservation stability of the second agent and the mixability of the first agent and the second agent.

Examples of ingredient (B) above used in the second agent of the present invention include, but are not particularly limited to, lauryl alcohol, myristyl alcohol, cetanol, stearyl alcohol, isostearyl alcohol, oleyl alcohol, arachyl alcohol, behenyl alcohol, octyldodecanol, and decyltetradecanol, and one or two or more types thereof may be contained. Of these, cetanol and stearyl alcohol are preferred from the viewpoint of improving preservation stability.

Two or more types of ingredient (B) above used in the second agent of the present invention are preferably contained from the viewpoint of improving preservation stability.

The content of ingredient (B) above used in the second agent of the present invention is not particularly limited, but ranges from preferably 0.6% to 7.2%, and more preferably 2.4% to 6% from the viewpoint of the preservation stability of the second agent and the mixability of the first agent and the second agent. If the content of ingredient (B) above is less than 0.6% or more than 7.2%, the mixability of the first agent and the second agent may deteriorate.

The second agent of the hair dye composition of the present invention contains hydrocarbon (C) that is in a liquid or paste form at 25° C. from the viewpoint of the preservation stability of the second agent and the mixability of the first agent and the second agent.

Among ingredient (C) above used in the second agent of the present invention, examples of the hydrocarbon that is in a liquid form at 25° C. include, but are not particularly limited to, paraffin, liquid paraffin, liquid isoparaffin, light liquid isoparaffin, heavy liquid isoparaffin, squalane, and vegetable squalane, and examples of the hydrocarbon that is in a paste form at 25° C. include, but are not particularly limited to, vaseline, and one or two or more types thereof may be contained. Of these, liquid paraffin, liquid isoparaffin, and squalane are preferred from the viewpoint of improving the preservation stability of the second agent, and liquid paraffin is more preferred from the viewpoint of improving the mixability of the first agent and the second agent.

The content of ingredient (C) above used in the second agent of the present invention is not particularly limited, but ranges from preferably 0.1% to 10%, and more preferably 1% to 8% from the viewpoint of the preservation stability of the second agent and the mixability of the first agent and the second agent. If the content of ingredient (C) above is less than 0.1%, the mixability of the first agent and the second agent may deteriorate. If the content of ingredient (C) above exceeds 10%, the preservation stability of the second agent and the mixability of the first agent and the second agent may deteriorate.

The second agent of the hair dye composition in the present invention contains an oxidizing agent from the viewpoint of decolorizing properties.

Examples of the oxidizing agent used in the second agent of the present invention include, but are not particularly limited to, hydrogen peroxide, urea peroxide, sodium percarbonate, potassium percarbonate, ammonium persulfate, sodium peroxide, potassium peroxide, magnesium peroxide, barium peroxide, and calcium peroxide, and one or two or more types thereof may be contained.

The content of the oxidizing agent used in the second agent of the present invention is not particularly limited, but preferably ranges from 0.1% to 6%.

The viscosity of the second agent of the present invention at 20° C. is not particularly limited, but the viscosity preferably ranges from 100 mPa·s to 100,000 mPa·s from the viewpoint of the mixability of the first agent and the second agent.

The viscosity of the second agent of the present invention at 20° C. was measured by filling a sample bottle (Food 140: Manufactured by Dai-ichi Glass Co., Ltd.) with 120 g of the first agent prepared by the usual method and allowing it to stand at 20° C. for 24 hours, and then rotating it for 1 minute: at 60 rpm using an M4 rotor when the viscosity was 100 mPa·s or more and less than 10,000 mPa·s as measured with a B-type viscometer (model: Digital Viscometer TVB-10M, manufactured by Toki Sangyo Co., Ltd.); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; and at 6 rpm using the M4 rotor when the viscosity was 50,000 mPa·s or more and 100,000 mPa·s or less as measured with the same.

The pH of the second agent of the present invention at 20° C. is not particularly limited, but preferably ranges from pH 2 to 4.

The pH of the second agent of the present invention at 20° C. was measured by filling a sample bottle (Food 140: Manufactured by DAI-ICHI GLASS Co., Ltd.) with 120 g of the second agent prepared by the usual method, allowing it to stand at 20° C. for 24 hours, and then measuring the pH of the stock solution with a glass electrode hydrogen ion concentration indicator (F-71, manufactured by HORIBA, Ltd.).

Examples of the formulation of the second agent of the present invention include, but are not particularly limited to, cream, emulsion and liquid. Of these, cream or emulsion is preferred from the viewpoint of usability.

Examples of the container to be filled with the second agent of the present invention include, but are not particularly limited to, tubes, bottles, jars, pouch films, and aerosols.

The hair dye composition in the present invention may, if necessary, contain one or two or more types of various ingredients selected from a cationic surfactant, vegetable oil, ester oil, wax, a fatty acid, silicone, a water-soluble polymer, a polyhydric alcohol, a chelating agent, an antioxidant, a pH adjuster, a preservative, a moisturizer, and the like other than ingredient (A) above and ingredient (B) above. Various ingredients other than ingredient (A) above and ingredient (B) above may be contained in the first agent, the second agent, or both the first agent and the second agent of the hair dye composition in the present invention.

The hair dye composition in the present invention may contain a cationic surfactant from the viewpoint of excellent combing with fingers upon rinsing hair.

Examples of the cationic surfactant used in the hair dye composition of the present invention include, but are not particularly limited to, lauryl trimethylammonium chloride, cetyltrimethylammonium chloride, stearyl trimethylammonium chloride, behenyl trimethylammonium chloride, and distearyldimethylammonium chloride, and one or two or more types thereof may be contained.

The content of the cationic surfactant used in the hair dye composition of the present invention is not particularly limited, but preferably ranges from 0.1% to 10%.

The hair dye composition in the present invention may contain vegetable oil.

Examples of the vegetable oil used in the hair dye composition of the present invention include, but are not particularly limited to, almond oil, avocado oil, argania spinosa kernel oil, Cananga odorata flower oil, olive oil, orange oil, apricot kernel oil, corn oil, sesame oil, pomegranate seed oil, shea butter, spearmint oil, damask rose flower oil, camellia oil, corn oil, Pelargonium graveolens flower oil, Nigella sativa seed oil, peppermint oil, Hypophaea rhamnoides seed oil, sunflower seed oil, grape seed oil, Plukenetia volubilis seed oil, Luffa cylindrica seed oil, bergamot fruit oil, jojoba oil, macadamia nut oil, mink oil, meadowfoam oil, peach kernel oil, coconut oil, eucalyptus oil, yucha oil, lavender oil, lemon fruit oil, rose hip oil, rosemary oil, and evening primrose oil, and one or two or more types thereof may be contained.

The content of the vegetable oil used in the hair dye composition of the present invention is not particularly limited, but preferably ranges from 0.1% to 20%.

The hair dye composition in the present invention may contain ester oil.

Examples of the ester oil used in the hair dye composition of the present invention include, but are not particularly limited to, diisopropyl adipate, diisobutyl adipate, bis(2-ethylhexyl) adipate, isopropyl myristate, cetyl ethyl hexanoate, cetyl isooctanoate, isononyl isononanoate, isodecyl isononanoate, isotridecyl isononanoate, diisopropyl sebacate, octyldodecyl myristate, isopropyl palmitate, hexyl laurate, oleyl oleate, decyl oleate, octyldodecyl oleate, oleyl erucate, octyldodecyl erucate, triisodecyl myristate, isostearyl myristate, 2-ethylhexyl palmitate, lauryl lactate, octyldodecyl lactate, isocetyl stearate, isocetyl isostearate, ethylene glycol di-2-ethylhexylate, glyceryl tricaprylate, and neopentyl glycol dicaprate, and one or two or more types thereof may be contained.

The content of the ester oil used in the hair dye composition of the present invention is not particularly limited, but preferably ranges from 0.1% to 20%.

The hair dye composition in the present invention may contain wax.

Examples of the wax used in the hair dye composition of the present invention include, but are not particularly limited to, microcrystalline wax, high melting point microcrystalline wax, synthetic hydrocarbon wax, polyethylene wax, synthetic wax, candelilla wax, rice bran wax, carnauba wax, beeswax, and sunflower seed wax, and one or two or more types thereof may be contained.

The content of the wax used in the hair dye composition of the present invention is not particularly limited, but preferably ranges from 0.1% to 20%.

The hair dye composition in the present invention may contain a fatty acid.

Examples of the fatty acid used in the hair dye composition of the present invention include, but are not particularly limited to, lauric acid, myristic acid, palmitic acid, stearic acid, behenic acid, isostearic acid, hydroxystearic acid, oleic acid, undecylenic acid, linoleic acid, ricinoleic acid, and lanolin fatty acid, and one or two or more types thereof may be contained.

The content of the fatty acid used in the hair dye composition of the present invention is not particularly limited, but preferably ranges from 0.1% to 10%.

The hair dye composition in the present invention may contain silicone from the viewpoint of excellent combing of finished hair with fingers.

Examples of the silicone used in the hair dye composition of the present invention include, but are not particularly limited to, decamethylcyclopentasiloxane, poly(oxyethylene-oxypropylene) methylpolysiloxane copolymer, polyoxyethylene-methylpolysiloxane copolymer, methylphenylpolysiloxane, methylpolysiloxane, and highly polymerized methylpolysiloxane, and one or two or more types thereof may be contained.

The content of the silicone used in the hair dye composition of the present invention is not particularly limited, but preferably ranges from 0.1% to 20%.

The hair dye composition in the present invention may contain a water-soluble polymer from the viewpoint of excellent combing of the hair with fingers upon rinsing.

Examples of the water-soluble polymer used in the hair dye composition of the present invention include, but are not particularly limited to, acrylamide-acrylic acid-dimethyldiallylammonium chloride copolymers, dimethyldiallylammonium chloride acrylamide copolymers, dimethyldiallylammonium chloride-acrylic acid copolymers, and poly(dimethyldimethylene pyrrolidinium chloride), and one or two or more types thereof may be contained.

The content of the water-soluble polymer used in the hair dye composition of the present invention is not particularly limited, but preferably ranges from 0.01% to 5%.

The hair dye composition in the present invention may contain a polyhydric alcohol from the viewpoint of hair dyeability.

Examples of the polyhydric alcohol used in the hair dye composition of the present invention include, but are not particularly limited to, glycerin, diglycerin, ethylene glycol, diethylene glycol, propylene glycol, dipropylene glycol, 1,3-butylene glycol, and polyethylene glycol, and one or two more types thereof may be contained.

The content of the polyhydric alcohol used in the hair dye composition of the present invention is not particularly limited, but preferably ranges from 0.1% to 10%.

The hair dye composition in the present invention may contain a chelating agent.

Examples of the chelating agent used in the hair dye composition of the present invention include, but are not particularly limited to, ethylenediaminetetraacetic acid tetrasodium salt tetrahydrate, pentasodium diethylenetriamine pentaacetate, and hydroxyethane diphosphonic acid, and one or two or more types thereof may be contained.

The content of the chelating agent used in the hair dye composition of the present invention is not particularly limited, but preferably ranges from 0.01% to 1%.

The hair dye composition in the present invention may contain an antioxidant.

Examples of the antioxidant used in the hair dye composition of the present invention include, but are not particularly limited to, sodium sulfite, L-ascorbic acid, and dl-α-tocopherol, and one or two or more types thereof may be contained.

The content of the antioxidant used in the hair dye composition of the present invention is not particularly limited, but preferably ranges from 0.1% to 5%.

The hair dye composition in the present invention may contain a pH adjuster.

Examples of the pH adjuster used in the hair dye composition of the present invention include, but are not particularly limited to, potassium hydroxide, sodium DL-malate, and magnesium sulfate, and one or two or more types thereof may be contained.

The content of the pH adjuster used in the hair dye composition of the present invention is not particularly limited, but preferably ranges from 0.01% to 5%.

The hair dye composition in the present invention may contain a preservative.

Examples of the preservative used in the hair dye composition of the present invention include, but are not particularly limited to, methyl paraoxibenzoate, propyl paraoxibenzoate, butyl paraoxibenzoate, phenoxyethanol, and ethylhexylglycerin, and one or two or more types thereof may be contained.

The content of the preservative used in the hair dye composition of the present invention is not particularly limited, but preferably ranges from 0.01% to 1%.

The hair dye composition in the present invention may contain a moisturizer.

Examples of the moisturizer used in the hair dye composition of the present invention include, but are not particularly limited to, an artichoke leaf extract, an ai leaf/stem extract, a red clover flower extract, an akebia stem extract, an Angelica keiskei extract, an ivyleaf morning glory callus culture extract, an arnica extract, an aloe extract, a strawberry seed extract, a dill seed extract, a turmeric extract, an edelweiss extract, a Fomes officinalis (mushroom) extract, a Scutellaria baicalensis root extract, a Cladosiphon okamuranus extract, a Panax ginseng root extract, a Lamium album extract, a Laminaria diabolica extract, a watercress extract, a Kjellmaniella crassifolia extract, a kamille extract, an Avena sativa (oat) kernel extract, an Artemisia capillaris extract, a licorice extract, a raspberry extract, a kiwi extract, a cucumber extract, a Sophora angustifolia root extract, an Alpinia speciosa leaf extract, a burdock root extract, a rice extract, a rice bran extract, an Eisenia arborea extract, a pomegranate fruit extract, a Prunus lannesiana flower extract, a common soapwort extract, a hawthorn extract, a Zanthoxylum piperitum peel extract, a shea butter extract, a Rehmannia glutinosa extract, a Perilla ocymoides leaf extract, a linden extract, a Filipendula extract, a peony extract, a ginger extract, a Tremella fuciformis (Mushroom) extract, a Caesalpinia sappan bark extract, an Equisetum arvense stem extract, a Porphyra yezoensis extract, an Urtica dioica leaf extract, an ivy extract, a Crataegus oxyacantha fruit extract, a Sambucus nigra extract, an Achillea millefolium flower extract, a Mentha piperrita extract, a sage extract, a Malva sylvestris (Mallow) flower extract, a Cnidium rhizome extract, a Swertia japonica extract, a jujube extract, a thyme extract, a Rosa damascena extract, a green tea extract, a Eugenia caryophyllus (clove) flower extract, a Citrus unshiu peel extract, a Camellia japonica flower extract, an Angelica acutiloba root extract, a Eucheuma serra/Grateloupia sparsa/Saccharina angustata/Ulva linza/Undaria pinnatifida extract, a tomato extract, a carrot extract, a garlic extract, a Nymphaea caerulea flower extract, a Rosa canina extract, a Nelumbium speciosum flower extract, a honey extract, a Hamamelis virginiana extract, a Parietaria officinalis extract, a beer extract, a Fucus vesiculosus extract, a sunflower seed extract, a Geranium robertianum extract, a Portulaca pilosa extract, a Vaccinium myrtillus extract, a Tussilago farfara leaf extract, a propolis extract, a pine extract, an Origanum majorana leaf extract, a marronnier extract, a mango fruit extract, a mangosteen pericarp extract, a Microcitrus australasica fruit extract, a Menyanthes trifoliata leaf extract, an evening primrose seed extract, a Terminalia catappa leaf extract, a peach fruit extract, peach fruit water, a peach leaf extract, a Saxifraga sarmentosa extract, a Citrus junos extract, a European birch leaf extract, a Coix lacryma-jobi ma-yuen seed extract, a peanut seedcoat extract, a Nephelium longana seed extract, an apple extract, a lettuce extract, a lemon grass leaf/stem extract, a rosemary extract, an Anthemis nobilis extract, a royal jelly extract, a logwood extract, a hydrolyzed okra seed extract, a hydrolyzed silk liquid, a hydrolyzed soybean extract, a seaweed extract, a brown algae extract, a black tea extract, a yeast extract, an Oryza sativa (rice) lees extract, a soy protein hydrolysate, collagen, and hyaluronic acid, and one or two or more types thereof may be contained.

The content of the moisturizer used in the hair dye composition of the present invention is not particularly limited, but preferably ranges from 0.01% to 10%.

The viscosity of the hair dye composition at 20° C. in the present invention is not particularly limited, but preferably ranges from 100 mPa·s to 100,000 mPa·s from the viewpoint of applicability.

The viscosity of the hair dye composition of the present invention at 20° C. was measured by mixing the first agent and the second agent, filling a sample bottle (Food 140: Manufactured by DAI-ICHI GLASS Co., Ltd.) with 120 g of the mixture, adjusting the temperature to 20° C., and then rotating it for 1 minute: at 60 rpm using an M4 rotor when the viscosity was 100 mPa·s or more and less than 10,000 mPa·s as measured with a B-type viscometer (Model: Digital Viscometer TVB-10M, manufactured by Toki Sangyo Co., Ltd); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; or at 6 rpm using the M4 rotor when the viscosity was 50,000 mPa·s or more and 100,000 mPa·s or less as measured with the same.

The pH of the hair dye composition at 20° C. in the present invention is not particularly limited, but preferably ranges from pH 8 to pH 12 from the viewpoint of hair dyeability.

The pH of the hair dye composition of the present invention at 20° C. was measured by mixing the first agent and the second agent, filling a sample bottle (Food 140: manufactured by DAI-ICHI GLASS Co., Ltd.) with 120 g of the mixture, adjusting the temperature to 20° C., and then measuring the pH of the hair dye composition with a glass electrode hydrogen ion concentration indicator (F-71, manufactured by the HORIBA, Ltd.).

The mixing ratio of the first agent and the second agent in the hair dye composition of the present invention is not particularly limited, but the mixing ratio of the first agent and the second agent ranges from preferably 1:3 to 3:1, more preferably 1:2 to 2:1, and is even more preferably 1:1.

Examples of the method for mixing the first agent and the second agent of the hair dye composition of the present invention include, but are not particularly limited to, a method that involves mixing the first agent and the second agent on a tray, a method that involves putting the first agent and the second agent into a container in which the two can be sealed and then shaking the container, a method that involves mixing the first agent and the second agent on the palm of a hand wearing a glove, and a method that involves dispensing the first agent and the second agent separately on a brush, and applying them while mixing them.

Examples of a tool for mixing the first agent and the second agent of the hair dye composition of the present invention include, but are not particularly limited to, a brush, a comb, a brush (hake), and a spatula.

Examples of a method for applying the hair dye composition of the present invention include, but are not particularly limited to, a method that involves taking the thus prepared hair dye composition by hand and applying it to the hair, a method that involves putting the prepared hair dye composition on a brush and then applying it to the hair, and a method that involves dispensing the prepared hair dye composition using a nozzle or a pump bottle for use.

### EXAMPLES

The present invention is described in more detail below with examples that do not limit the present invention in any way.

An evaluation test presented herein was conducted by varying the ingredients contained in the first agent and the second agent of the hair dye composition, as well as their contents. All units indicating the content of each ingredient are all mass %, and each composition was prepared by a usual method.

### <Preservation Stability of the First Agent>

In the evaluation test concerning “the preservation stability of the first agent” described herein, a 140-g sample bottle (Food 140: Manufactured by Dai-ichi Glass Co., Ltd.) was filled with 120 g of the first agent prepared by a usual method, and then stored under 40° C. conditions for 3 months. The appearance was visually confirmed to evaluate preservation stability based on the formation of water droplets by the first agent (the state in which water droplets can be confirmed on the surface) and the separation (the state in which the first agent is clearly separated into two or more layers), as described below.

### <Evaluation Criteria for the Preservation Stability of the First Agent>

A: No change was confirmed for the preparation after 3 months of storage under 40° C. conditions.
B: No change was confirmed for the preparation after 1 month of storage under 40° C. conditions, but the formation of water droplets was slightly confirmed for the preparation after 3 months of storage.
C: Separation of the preparation was confirmed after less than 1 month of storage under 40° C. conditions.
-: Due to separation immediately after preparation of the preparation, no evaluation could be made.

### <Preservation Stability of the Second Agent>

In the evaluation test concerning “the preservation stability of the second agent” described herein, a 140-g sample bottle (Food 140: Manufactured by Dai-ichi Glass Co., Ltd.) was filled with 120 g of the second agent prepared by a usual method, and then stored under 40° C. conditions for 3 months. The appearance was visually confirmed to evaluate preservation stability based on the formation of water droplets by the second agent (the state in which water droplets can be confirmed on the surface) and the separation (the state in which the second agent is clearly separated into two or more layers), as described below.

### <Evaluation Criteria for the Preservation Stability of the Second Agent>

A: No change was confirmed for the composition after 3 months of storage under 40° C. conditions.
B: No change was confirmed for the composition after 1 month of storage under 40° C. conditions, but the formation of water droplets was slightly confirmed for the composition after 3 months of storage.
C: Separation of the composition was confirmed after less than 1 month of storage under 40° C. conditions.
-: Due to separation immediately after preparation of the preparation, no evaluation could be made.

### <Mixability of the First Agent and the Second Agent>

In the evaluation test concerning “the mixability of the first agent and the second agent” described herein, the first agent and the second agent of the hair dye composition were left to stand at 20° C. for 1 day to adjust the temperature, 40 g of the first agent and 40 g of the second agent were weighed in a 200-mL Griffin beaker. The Griffin beaker was tilted at a 30-degree angle from the vertical position and stirred in a circular motion with a glass rod. The evaluation was made as described below based on the number of stirring required for the uniform mixing of the mixture.

### <Evaluation Method for the Mixability of the First Agent and the Second Agent>

A: Mixed uniformly by less than 50 times of stirring.
B: Mixed uniformly by less than 100 times of stirring.
C: Not uniformly mixed even after 100 times of stirring, and lumps and remaining mixtures were confirmed.
-: Due to separation immediately after preparation of the first agent or the second agent, no evaluation could be made.

### (First Evaluation Test)

In the first evaluation test, evaluation was made by varying the content and the type of ingredient (A) in the first agent of the hair dye composition prepared by the usual method. Table 1 lists the ingredients, contents and evaluation results of the first agent and the second agent.

TABLE 1

| | | Exam- | Exam- | Exam- | Exam- | Exam- | Exam- | Exam- | Exam- | Comparative |
|---|---|---|---|---|---|---|---|---|---|---|
| Ingredient name (First agent) | | ple 1 | ple 2 | ple 3 | ple 4 | ple 5 | ple 6 | ple 7 | ple 8 | example 1 |
| (A) | Polyoxyethylene (15) cetyl ether | 0.80 | 1.40 | 2.00 | 0.80 | 2.80 | 3.60 | 3.00 | — | — |
| (A) | Polyoxyethylene (40) cetyl ether | 0.40 | 0.70 | 1.00 | 2.20 | 1.40 | 1.80 | — | 3.00 | — |
| (B) | Cetanol | 1.20 | 1.20 | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 | 4.20 | 4.20 |
| (C) | Liquid paraffin | 3.00 | 3.00 | 3.00 | 3.00 | 3.00 | 3.00 | 3.00 | 3.00 | 3.00 |
| | Monoethanolamine | 6.40 | 6.40 | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 | 0.05 | 0.05 |
| | 2,4-Diaminophenoxyethanol | 0.05 | 0.05 | 0.05 | 0.05 | 0.05 | 0.05 | 0.05 | 0.05 | 0.05 |
| | hydrochloride | | | | | | | | | |
| | Resorcin | 0.80 | 0.80 | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 | 0.30 | 0.30 |
| | Behenyltrimethylammonium | 1.60 | 1.60 | 1.60 | 1.60 | 1.60 | 1.60 | 1.60 | 1.60 | 1.60 |
| | chloride | | | | | | | | | |
| | Methylpolysiloxane | 2.00 | 2.00 | 2.00 | 2.00 | 2.00 | 2.00 | 2.00 | 2.00 | 2.00 |
| | Poly N,N-dimethyl- | 0.40 | 0.40 | 0.40 | 0.40 | 0.40 | 0.40 | 0.40 | 0.40 | 0.40 |
| | 3,4-dimethylene- | | | | | | | | | |
| | pyrrolidinium chloride | | | | | | | | | |
| | Pentasodium diethylenetriamine | 0.20 | 0.20 | 0.20 | 0.20 | 0.20 | 0.20 | 0.20 | 0.20 | 0.20 |
| | pentaacetate | | | | | | | | | |
| | Sodium sulfite | 0.20 | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 | 0.50 |
| | Purified water | 76.10 | 75.20 | 74.30 | 74.30 | 73.10 | 71.90 | 74.30 | 74.30 | 77.30 |
| | Total | 100.00 | 100.00 | 100.00 | 100.00 | 100.00 | 100.00 | 100.00 | 100.00 | 100.00 |
| | | Exam- | Exam- | Exam- | Exam- | Exam- | Exam- | Exam- | Exam- | Comparative |
| Ingredient name (Second agent) | | ple 1 | ple 2 | ple 3 | ple 4 | ple 5 | ple 6 | ple 7 | ple 8 | example 1 |
| (A) | Polyoxyethylene (10) cetyl ether | 1.00 | 1.00 | 1.00 | 1.00 | 1.00 | 1.00 | 1.00 | 1.00 | 1.00 |
| (A) | Polyoxyethylene (20) cetyl ether | 1.60 | 1.60 | 1.60 | 1.60 | 1.60 | 1.60 | 1.60 | 1.60 | 1.60 |
| (B) | Cetanol | 3.60 | 3.60 | 3.60 | 3.60 | 3.60 | 3.60 | 3.60 | 3.60 | 3.60 |
| (B) | Stearyl alcohol | 1.20 | 1.20 | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 | 0.14 | 0.14 |
| | acid solution | | | | | | | | | |
| | Potassium hydroxide | 0.05 | 0.05 | 0.05 | 0.05 | 0.05 | 0.05 | 0.05 | 0.05 | 0.05 |
| | Purified water | 83.10 | 83.10 | 83.10 | 83.10 | 83.10 | 83.10 | 83.10 | 83.10 | 83.10 |
| | Total | 100.00 | 100.00 | 100.00 | 100.00 | 100.00 | 100.00 | 100.00 | 100.00 | 100.00 |
| Eval- | Preservation stability | B | A | A | A | A | A | B | B | — |
| uation | of first agent (40° C.) | | | | | | | | | |
| items | Preservation stability | A | A | A | A | A | A | A | A | A |
| | of second apent (40° C.) | | | | | | | | | |
| | Mixability of first agent | B | A | A | A | A | B | A | A | — |
| | and second agent | | | | | | | | | |

From Examples 1 to 8 shown in Table 1, good results were obtained for the preservation stability of the first agent and the mixability of the first agent and the second agent.

### (Second Evaluation Test)

In the second evaluation test, evaluation was made by varying the content and the type of ingredient (B) in the first agent of the hair dye composition prepared by the usual method. Table 2 lists the ingredients, contents and evaluation results of the first agent and the second agent.

TABLE 2

| | | Exam- | Exam- | Exam- | Exam- | Exam- | Exam- | Comparative |
|---|---|---|---|---|---|---|---|---|
| Ingredient name (First agent) | | ple 9 | ple 10 | ple 11 | ple 12 | ple 13 | ple 14 | example 2 |
| (A) | Polyoxyethylene (15) cetyl ether | 2.00 | 2.00 | 2.00 | 2.00 | 2.00 | 2.00 | 2.00 |
| (A) | Polyoxyethylene (40) cetyl ether | 1.00 | 1.00 | 1.00 | 1.00 | 1.00 | 1.00 | 1.00 |
| (B) | Cetanol | 0.60 | 0.80 | 1.40 | 1.60 | 5.40 | — | — |
| (B) | Stearyl alcohol | 2.10 | 2.80 | 4.90 | 5.60 | — | 5.40 | — |
| (C) | Liquid paraffin | 3.00 | 3.00 | 3.00 | 3.00 | 3.00 | 3.00 | 3.00 |
| | Monoethanolamine | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 |
| | 2,4-Diaminophenoxyethanol | 0.05 | 0.05 | 0.05 | 0.05 | 0.05 | 0.05 | 0.05 |
| | hydrochloride | | | | | | | |
| | Resorcin | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 |
| | Behenyltrimethylammonium chloride | 1.60 | 1.60 | 1.60 | 1.60 | 1.60 | 1.60 | 1.60 |
| | Methylpolysiloxane | 2.00 | 2.00 | 2.00 | 2.00 | 2.00 | 2.00 | 2.00 |
| | Poly N,N-dimethyl-3,4-dimethylene- | 0.40 | 0.40 | 0.40 | 0.40 | 0.40 | 0.40 | 0.40 |
| | pyrrolidinium chloride | | | | | | | |
| | Pentasodium diethylenetriamine | 0.20 | 0.20 | 0.20 | 0.20 | 0.20 | 0.20 | 0.20 |
| | pentaacetate | | | | | | | |
| | Sodium sulfite | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 |
| | Purified water | 77.00 | 76.10 | 73.40 | 72.50 | 74.30 | 74.30 | 79.70 |
| | Total | 100.00 | 100.00 | 100.00 | 100.00 | 100.00 | 100.00 | 100.00 |
| | | Exam- | Exam- | Exam- | Exam- | Exam- | Exam- | Comparative |
| Ingredient name (Second agent) | | ple 9 | ple 10 | ple 11 | ple 12 | ple 13 | ple 14 | example 2 |
| (A) | Polyoxyethylene (10) cetyl ether | 1.00 | 1.00 | 1.00 | 1.00 | 1.00 | 1.00 | 1.00 |
| (A) | Polyoxyethylene (20) cetyl ether | 1.60 | 1.60 | 1.60 | 1.60 | 1.60 | 1.60 | 1.60 |
| (B) | Cetanol | 3.60 | 3.60 | 3.60 | 3.60 | 3.60 | 3.60 | 3.60 |
| (B) | Stearyl alcohol | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 |
| | acid solution | | | | | | | |
| | Potassium hydroxide | 0.05 | 0.05 | 0.05 | 0.05 | 0.05 | 0.05 | 0.05 |
| | Purified water | 83.10 | 83.10 | 83.10 | 83.10 | 83.10 | 83.10 | 83.10 |
| | Total | 100.00 | 100.00 | 100.00 | 100.00 | 100.00 | 100.00 | 100.00 |
| Eval- | Preservation stability of first agent (40° C.) | A | A | A | A | B | B | — |
| uation | Preservation stability of second agent (40° C.) | A | A | A | A | A | A | A |
| items | Mixability of first agent and second agent | B | A | A | B | A | A | — |

From Examples 9 to 14 shown in Table 2, good results were obtained for the preservation stability of the first agent and the mixability of the first agent and the second agent.

### (Third Evaluation Test)

In the third evaluation test, evaluation was made by varying the content and the type of ingredient (C) in the first agent of the hair dye composition prepared by the usual method. Table 3 lists the ingredients, contents and evaluation results of the first gent and the second agent.

TABLE 3

| | | Exam- | Exam- | Exam- | Exam- | Exam- | Exam- | Exam- | Comparative |
|---|---|---|---|---|---|---|---|---|---|
| Ingredient name (First agent) | | ple 15 | ple 16 | ple 17 | ple 18 | ple 19 | ple 20 | ple 21 | example 3 |
| (A) | Polyoxyethylene (15) cetyl ether | 2.00 | 2.00 | 2.00 | 2.00 | 2.00 | 2.00 | 2.00 | 2.00 |
| (A) | Polyoxyethylene (40) cetyl ether | 1.00 | 1.00 | 1.00 | 1.00 | 1.00 | 1.00 | 1.00 | 1.00 |
| (B) | Cetanol | 1.20 | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 | 4.20 |
| (C) | Liquid paraffin | 0.10 | 1.00 | 8.00 | 10.00 | — | — | — | — |
| (C) | Liquid isoparaffin | — | — | — | — | 3.00 | — | — | — |
| (C) | Squalane | — | — | — | — | — | 3.00 | — | — |
| (C) | Vaseline | | | | | | | 3.00 | |
| | Monoethanolamine | 6.40 | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 | 0.05 |
| | 2,4-Diaminophenoxyethanol | 0.05 | 0.05 | 0.05 | 0.05 | 0.05 | 0.05 | 0.05 | 0.05 |
| | hydrochloride | | | | | | | | |
| | Resorcin | 0.80 | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 | 0.30 |
| | Behenyltrimethylammonium chloride | 1.60 | 1.60 | 1.60 | 1.60 | 1.60 | 1.60 | 1.60 | 1.60 |
| | Methylpolysiloxane | 2.00 | 2.00 | 2.00 | 2.00 | 2.00 | 2.00 | 2.00 | 2.00 |
| | Poly N,N-dimethyl-3,4-dimethylene- | 0.40 | 0.40 | 0.40 | 0.40 | 0.40 | 0.40 | 0.40 | 0.40 |
| | pyrrolidinium chloride | | | | | | | | |
| | Pentasodium diethylenetriamine | 0.20 | 0.20 | 0.20 | 0.20 | 0.20 | 0.20 | 0.20 | 0.20 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 |
| | Purified water | 77.20 | 76.30 | 69.30 | 67.30 | 74.30 | 74.30 | 74.30 | 77.30 |
| | Total | 100.00 | 100.00 | 100.00 | 100.00 | 100.00 | 100.00 | 100.00 | 100.00 |
| | | Exam- | Exam- | Exam- | Exam- | Exam- | Exam- | Exam- | Comparative |
| Ingredient name (Second agent) | | ple 15 | ple 16 | ple 17 | ple 18 | ple 19 | ple 20 | ple 21 | example 3 |
| (A) | Polyoxyethylene (10) cetyl ether | 1.00 | 1.00 | 1.00 | 1.00 | 1.00 | 1.00 | 1.00 | 1.00 |
| (A) | Polyoxyethylene (20) cetyl ether | 1.60 | 1.60 | 1.60 | 1.60 | 1.60 | 1.60 | 1.60 | 1.60 |
| (B) | Cetanol | 3.60 | 3.60 | 3.60 | 3.60 | 3.60 | 3.60 | 3.60 | 3.60 |
| (B) | Stearyl alcohol | 1.20 | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 | 0.14 |
| | acid solution | | | | | | | | |
| | Potassium hydroxide | 0.05 | 0.05 | 0.05 | 0.05 | 0.05 | 0.05 | 0.05 | 0.05 |
| | Purified water | 83.10 | 83.10 | 83.10 | 83.10 | 83.10 | 83.10 | 83.10 | 83.10 |
| | Total | 100.00 | 100.00 | 1 00.00 | 100.00 | 100.00 | 100.00 | 100.00 | 100.00 |
| Eval- | Preservation stability of | A | A | A | B | A | A | B | A |
| uation | first agent (40° C.) | | | | | | | | |
| items | Preservation stability of | A | A | A | A | A | A | A | A |
| | second agent (40° C.) | | | | | | | | |
| | Mixability of first agent | B | A | A | B | B | B | B | C |
| | and second agent | | | | | | | | |

From Examples 15 to 21 shown in Table 3, good results were obtained for the preservation stability of the first agent and the mixability of the first agent and the second agent.

### (Fourth Evaluation Test)

In the fourth evaluation test, evaluation was made by varying the content and the type of ingredient (A) in the second agent of the hair dye composition prepared by the usual method. Table 4 lists the ingredients, contents and evaluation results of the first agent and the second agent.

TABLE 4

| | | Exam- | Exam- | Exam- | Exam- | Exam- | Exam- | Exam | Comparative |
|---|---|---|---|---|---|---|---|---|---|
| Ingredient name (First agent) | | ple 22 | ple 23 | ple 24 | ple 25 | ple 26 | ple 27 | ple 28 | example 4 |
| (A) | Polyoxyethylene (15) cetyl ether | 2.00 | 2.00 | 2.00 | 2.00 | 2.00 | 2.00 | 2.00 | 2.00 |
| (A) | Polyoxyethylene (40) cetyl ether | 1.00 | 1.00 | 1.00 | 1.00 | 1.00 | 1.00 | 1.00 | 1.00 |
| (B) | Cetanol | 1.20 | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 | 4.20 |
| (C) | Liquid paraffin | 3.00 | 3.00 | 3.00 | 3.00 | 3.00 | 3.00 | 3.00 | 3.00 |
| | Monoethanolamine | 6.40 | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 | 0.05 |
| | 2,4-Diaminophenoxyethanol | 0.05 | 0.05 | 0.05 | 0.05 | 0.05 | 0.05 | 0.05 | 0.05 |
| | hxdrochloride | | | | | | | | |
| | Resorcin | 0.80 | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 | 0.30 |
| | Behenyltrimethylammonium chloride | 1.60 | 1.60 | 1.60 | 1.60 | 1.60 | 1.60 | 1.60 | 1.60 |
| | Methylpolysiloxane | 2.00 | 2.00 | 2.00 | 2.00 | 2.00 | 2.00 | 2.00 | 2.00 |
| | Poly N,N-dimethyl-3,4-dimethylene- | 0.40 | 0.40 | 0.40 | 0.40 | 0.40 | 0.40 | 0.40 | 0.40 |
| | pyrrolidinium chloride | | | | | | | | |
| | Pentasodium diethylenetriamine | 0.20 | 0.20 | 0.20 | 0.20 | 0.20 | 0.20 | 0.20 | 0.20 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 |
| | Purified water | 74.30 | 74.30 | 74.30 | 74.30 | 74.30 | 74.30 | 74.30 | 74.30 |
| | Total | 100.00 | 100.00 | 100.00 | 100.00 | 100.00 | 100.00 | 100.00 | 100.00 |
| | | Exam- | Exam- | Exam- | Exam- | Exam- | Exam- | Exam | Comparative |
| Ingredient name (Second agent) | | ple 22 | ple 23 | ple 24 | ple 25 | ple 26 | ple 27 | ple 28 | example 4 |
| (A) | Polyoxyethylene (10) cetyl ether | 0.10 | 0.50 | 1.30 | 1.60 | — | 2.60 | — | — |
| (A) | Polyoxyethylene (20) cetyl ether | 0.16 | 0.80 | 2.10 | 2.60 | — | — | 2.60 | — |
| (A) | Polyoxyethylene (20) stearyl ether | — | — | — | — | 1.00 | — | — | — |
| (A) | Polyoxyethylene (30) stearyl ether | — | — | — | — | 1.60 | — | — | — |
| (B) | Cetanol | 3.60 | 3.60 | 3.60 | 3.60 | 3.60 | 3.60 | 3.60 | 3.60 |
| (B) | Stearyl alcohol | 1.20 | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 | 5.81 |
| | Liquid paraffin | 2.00 | 2.00 | 2.00 | 2.00 | 2.00 | 2.00 | 2.00 | 2.00 |
| | Polyethylene glycol 400 | 1.50 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 | 0.14 |
| | acid solution | | | | | | | | |
| | Potassium hydroxide | 0.05 | 0.05 | 0.05 | 0.05 | 0.05 | 0.05 | 0.05 | 0.05 |
| | Purified water | 83.44 | 82.40 | 80.30 | 79.50 | 81.10 | 81.10 | 81.10 | 83.70 |
| | Total | 100.00 | 100.00 | 100.00 | 100.00 | 100.00 | 100.00 | 100.00 | 100.00 |
| Eval- | Preservation stability of | A | A | A | A | A | A | A | A |
| uation | first agent (40° C.) | | | | | | | | |
| items | Preservation stability of | B | A | A | A | A | B | B | — |
| | second agent (40° C.) | | | | | | | | |
| | Mixability of first agent | B | A | A | B | A | A | A | — |
| | and second agent | | | | | | | | |

From Examples 22 to 28 shown in Table 4, good results were obtained for the preservation stability of the second agent and the mixability of the first agent and the second agent.

### (Fifth Evaluation Test)

In the fifth evaluation test, evaluation was made by varying the content and the type of ingredient (B) in the second agent of the hair dye composition prepared by the usual method. Table 5 lists the ingredients, contents and evaluation results of the first agent and the second agent.

TABLE 5

| | | Exam- | Exam- | Exam- | Exam- | Exam- | Exam- | Comparative |
|---|---|---|---|---|---|---|---|---|
| Ingredient name (First agent) | | ple 29 | ple 30 | ple 31 | ple 32 | ple 33 | ple 34 | example 5 |
| (A) | Polyoxyethylene (15) cetyl ether | 2.00 | 2.00 | 2.00 | 2.00 | 2.00 | 2.00 | 2.00 |
| (A) | Polyoxyethylene (40) cetyl ether | 1.00 | 1.00 | 1.00 | 1.00 | 1.00 | 1.00 | 1.00 |
| (B) | Cetanol | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 |
| (C) | Liquid paraffin | 3.00 | 3.00 | 3.00 | 3.00 | 3.00 | 3.00 | 3.00 |
| | Monoethanolamine | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 |
| | 2.4-Diaminophenoxyethanol | 0.05 | 0.05 | 0.05 | 0.05 | 0.05 | 0.05 | 0.05 |
| | hydrochloride | | | | | | | |
| | Resorcin | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 |
| | Behenyltrimethylammonium chloride | 1.60 | 1.60 | 1.60 | 1.60 | 1.60 | 1.60 | 1.60 |
| | Methylpolysiloxane | 2.00 | 2.00 | 2.00 | 2.00 | 2.00 | 2.00 | 2.00 |
| | Poly N,N-dimethyl-3,4-dimethylene- | 0.40 | 0.40 | 0.40 | 0.40 | 0.40 | 0.40 | 0.40 |
| | pyrrolidinium chloride | | | | | | | |
| | Pentasodium diethylenetriamine | 0.20 | 0.20 | 0.20 | 0.20 | 0.20 | 0.20 | 0.20 |
| | pentaacetate | | | | | | | |
| | Sodium sulfite | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 |
| | Purified water | 74.30 | 74.30 | 74.30 | 74.30 | 74.30 | 74.30 | 74.30 |
| | Total | 100.00 | 100.00 | 100.00 | 100.00 | 100.00 | 100.00 | 100.00 |
| | | Exam- | Exam- | Exam- | Exam- | Exam- | Exam- | Comparative |
| Ingredient name (Second agent) | | ple 29 | ple 30 | ple 31 | ple 32 | ple 33 | ple 34 | example 5 |
| (A) | Polyoxyethylene (10) cetyl ether | 1.00 | 1.00 | 1.00 | 1.00 | 1.00 | 1.00 | 1.00 |
| (A) | Polyoxyethylene (20) cetyl ether | 1.60 | 1.60 | 1.60 | 1.60 | 1.60 | 1.60 | 1.60 |
| (B) | Cetanol | 0.45 | 1.80 | 4.50 | 5.40 | 4.80 | — | — |
| (B) | Stearyl alcohol | 0.15 | 0.60 | 1.50 | 1.80 | — | 4.80 | — |
| (C) | Liquid paraffin | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 |
| | acid solution | | | | | | | |
| | Potassium hydroxide | 0.05 | 0.05 | 0.05 | 0.05 | 0.05 | 0.05 | 0.05 |
| | Purified water | 87.30 | 85.50 | 81.90 | 80.70 | 83.10 | 83.10 | 87.90 |
| | Total | 100.00 | 100.00 | 100.00 | 100.00 | 100.00 | 100.00 | 100.00 |
| Eval- | Preservation stability of | A | A | A | A | A | A | A |
| uation | first agent (40° C.) | | | | | | | |
| items | Preservation stability of | A | A | A | A | B | B | — |
| | second agent (40° C.) | | | | | | | |
| | Mixability of first agent | B | A | A | B | A | A | — |
| | and second agent | | | | | | | |

From Examples 29 to 34 shown in Table 5, good results were obtained for the preservation stability of the second agent and the mixability of the first agent and the second agent.

### (Sixth Evaluation Test)

In the sixth evaluation test, evaluation was made by varying the content and the type of ingredient (C) in the second agent of the hair dye composition prepared by the usual method. Table 6 lists the ingredients, contents and evaluation results of the first agent and the second agent.

TABLE 6

| | | Exam- | Exam- | Exam- | Exam- | Exam- | Exam- | Exam- | Comparative |
|---|---|---|---|---|---|---|---|---|---|
| Ingredient name (First agent) | | ple 35 | ple 36 | ple 37 | ple 38 | ple 39 | ple40 | ple 41 | example 6 |
| (A) | Polyoxyethylene (15) cetyl ether | 2.00 | 2.00 | 2.00 | 2.00 | 2.00 | 2.00 | 2.00 | 2.00 |
| (A) | Polyoxyethylene (40) cetyl ether | 1.00 | 1.00 | 1.00 | 1.00 | 1.00 | 1.00 | 1.00 | 1.00 |
| (B) | Cetanol | 1.20 | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 | 4.20 |
| (C) | Liquid paraffin | 3.00 | 3.00 | 3.00 | 3.00 | 3.00 | 3.00 | 3.00 | 3.00 |
| | Monoethanolamine | 6.40 | 6.40 | 6.40 | 6.40 | 6.40 | 6.40 | 6.40 | 6.40 |
| | Toluene-2,5-diamine | 1.00 | 1.00 | 1.00 | 1.00 | 1.00 | 1.00 | 1.00 | 1.00 |
| | Para-aminophenol | 0.30 | 0.30 | 0.30 | 0.30 | 0.30 | 0.30 | 0.30 | 0.30 |
| | Paraphenylenediamine | 0.50 | 0.50 | 0.50 | 0.50 | 0.50 | 0.50 | 0.50 | 0.50 |
| | Meta-aminophenol | 0.05 | 0.05 | 0.05 | 0.05 | 0.05 | 0.05 | 0.05 | 0.05 |
| | 2,4-Diaminophenoxyethanol | 0.05 | 0.05 | 0.05 | 0.05 | 0.05 | 0.05 | 0.05 | 0.05 |
| | hydrochloride | | | | | | | | |
| | Resorcin | 0.80 | 0.80 | 0.80 | 0.80 | 0.80 | 0.80 | 0.80 | 0.80 |
| | 5-Amino ortho cresol | 0.30 | 0.30 | 0.30 | 0.30 | 0.30 | 0.30 | 0.30 | 0.30 |
| | Behenyltrimethylammonium chloride | 1.60 | 1.60 | 1.60 | 1.60 | 1.60 | 1.60 | 1.60 | 1.60 |
| | Methylpolysiloxane | 2.00 | 2.00 | 2.00 | 2.00 | 2.00 | 2.00 | 2.00 | 2.00 |
| | Poly N,N-dimethyl-3,4-dimethylene- | 0.40 | 0.40 | 0.40 | 0.40 | 0.40 | 0.40 | 0.40 | 0.40 |
| | pyrrolidinium chloride | | | | | | | | |
| | Pentasodium diethylenetriamine | 0.20 | 0.20 | 0.20 | 0.20 | 0.20 | 0.20 | 0.20 | 0.20 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 |
| | Purified water | 74.30 | 74.30 | 74.30 | 74.30 | 74.30 | 74.30 | 74.30 | 74.30 |
| | Total | 100.00 | 100.00 | 100.00 | 100.00 | 100.00 | 100.00 | 100.00 | 100.00 |
| | | Exam- | Exam- | Exam- | Exam- | Exam- | Exam- | Exam- | Comparative |
| Ingredient name (Second agent) | | ple 35 | ple 36 | ple 37 | ple 38 | ple 39 | ple40 | ple 41 | example 6 |
| (A) | Polyoxyethylene (10) cetyl ether | 1.00 | 1.00 | 1.00 | 1.00 | 1.00 | 1.00 | 1.00 | 1.00 |
| (A) | Polyoxyethylene (20) cetyl ether | 1.60 | 1.60 | 1.60 | 1.60 | 1.60 | 1.60 | 1.60 | 1.60 |
| (B) | Cetanol | 3.60 | 3.60 | 3.60 | 3.60 | 3.60 | 3.60 | 3.60 | 3.60 |
| (B) | Stearyl alcohol | 1.20 | 1.20 | 1.20 | 1.20 | 1.20 | 1.20 | 1.20 | 1.20 |
| (C) | Liquid paraffin | 0.10 | 1.00 | 8.00 | 10.00 | — | — | — | — |
| (C) | Liquid isoparaffin | — | — | — | — | 2.00 | — | — | — |
| (C) | Squalane | — | — | — | — | — | 2.00 | — | — |
| (C) | Vaseline | — | — | — | — | — | — | 2.00 | — |
| | Hydrogen peroxide | 5.81 | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 | 1.50 |
| | Hydroxyethane diphosphonic | 0.14 | 0.14 | 0.14 | 0.14 | 0.14 | 0.14 | 0.14 | 0.14 |
| | acid solution | | | | | | | | |
| | Potassium hydroxide | 0.05 | 0.05 | 0.05 | 0.05 | 0.05 | 0.05 | 0.05 | 0.05 |
| | Purified water | 85.00 | 84.10 | 77.10 | 75.10 | 83.10 | 83.10 | 83.10 | 85.10 |
| | Total | 100.00 | 100.00 | 100.00 | 100.00 | 100.00 | 100.00 | 100.00 | 100.00 |
| Eval- | Preservation stability of | A | A | A | A | A | A | A | A |
| uation | first agent (40° C.) | | | | | | | | |
| items | Preservation stability of | A | A | A | B | A | A | B | A |
| | second agent (40° C.) | | | | | | | | |
| | Mixability of first agent | B | A | A | B | B | B | B | C |
| | and second agent | | | | | | | | |

From Examples 35 to 41 shown in Table 6, good results were obtained for the preservation stability of the second agent and the mixability of the first agent and the second agent.

Table 7 lists the results of the first agents and the second agents of the hair dye compositions prepared by the usual method in Examples 42 to 49.

The first agents and the second agents obtained according to Examples 42 to 49 exhibited good results with respect to the preservation stability of the first agents, the preservation stability of the second agents, and the mixability of the first agents and the second agents. Note that in Example 49, 30 g of the first agent and 60 g of the second agent were mixed and then the mixability of the first agent and the second agent was evaluated.

TABLE 7

| | | Exam- | Exam- | Exam- | Exam- | Exam- | Exam- | Exam- | Exam- |
|---|---|---|---|---|---|---|---|---|---|
| Ingredient name (First agent) | | ple 42 | ple 43 | ple 44 | ple 45 | ple 46 | ple 47 | ple 48 | ple 49 |
| (A) | Polyoxyethylene (13) cetyl ether | 0.18 | 0.18 | 0.18 | 0.18 | 0.18 | 0.18 | 0.18 | — |
| (A) | Polvoxyethylene (15) cetyl ether | 2.00 | 2.00 | 0.80 | 0.80 | 0.80 | 0.80 | 0.80 | 0.80 |
| (A) | Polyoxyethylene (40) cetyl ether | 1.00 | 1.00 | 2.20 | 2.20 | 2.20 | 2.20 | 2.20 | 2.20 |
| (B) | Cetanol | 1.20 | 1.20 | 1.20 | 1.20 | 1.20 | 1.20 | 1.20 | 1.20 |
| (B) | Stearyl alcohol | 4.20 | 4.20 | 4.20 | 4.20 | 4.20 | 4.20 | 4.20 | 4.20 |
| (C) | Liquid paraffin | 3.00 | 3.00 | 3.00 | 3.00 | 3.00 | 3.00 | 3.00 | 3.00 |
| | Monoethanolamine | 6.40 | 6.40 | 4.40 | 3.60 | 3.60 | 3.60 | 3.60 | 4.80 |
| | Toluene-2,5-diamine | 0.28 | 0.86 | 1.17 | 1.52 | 1.52 | 2.10 | 3.18 | — |
| | Para-aminophenol | 1.00 | 1.00 | 0.97 | 0.33 | 0.33 | 0.51 | — | — |
| | Paraphenylenediamine | — | — | — | — | — | — | — | 1.15 |
| | Meta-aminophenol | — | 0.05 | 0.21 | 0.19 | 0.19 | 0.33 | 0.30 | |
| | 2,4-Diaminophenoxyethanol hydrochloride | — | — | — | 0.06 | 0.06 | 0.12 | 0.10 | 0.60 |
| | Resorcin | 0.56 | 0.54 | 0.84 | 0.66 | 0.66 | 0.89 | 1.20 | 0.45 |
| | 5-Amino ortho creso | 0.24 | 0.24 | 0.18 | 0.12 | 0.12 | 0.04 | 0.08 | — |
| | Behenyltrimethylammonium chloride | 1.60 | 1.60 | 1.60 | 1.60 | 1.60 | 1.60 | 1.60 | 1.60 |
| | Camellia oil | 0.01 | 0.01 | 0.0 | 0.01 | 0.01 | 0.01 | 0.01 | 0.01 |
| | Methylpolysiloxane | 3.62 | 3.62 | 3.62 | 3.62 | 3.62 | 3.62 | 3.62 | 2.00 |
| | Highly polymerized methylpolysiloxane | 0.18 | 0.18 | 0.18 | 0.18 | 0.18 | 0.18 | 0.18 | — |
| | Poly N,N-dimethyl-3,4-dimethylene | 0.42 | 0.42 | 0.42 | 0.42 | 0.42 | 0.42 | 0.42 | 0.42 |
| | pyrrolidinium chloride | | | | | | | | |
| | Ethanol | 0.40 | 0.40 | 0.40 | 0.40 | 0.40 | 0.40 | 0.40 | 0.40 |
| | Glycerin | 0.09 | 0.09 | 0.09 | 0.09 | 0.09 | 0.09 | 0.09 | — |
| | 1,3-Butylene glycol | 0.01 | 0.01 | 0.01 | 0.01 | 0.01 | 0.01 | 0.01 | 0.01 |
| | Pentasodium diethylenetriamine | 0.20 | 0.20 | 0.20 | 0.92 | 0.92 | 0.92 | 0.92 | 0.92 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.52 | 0.55 | 0.57 | 0.59 | 0.59 | 0.62 | 0.68 | 0.50 |
| | dl-α-Tocopherol | 0.01 | 0.01 | 0.01 | 0.01 | 0.01 | 0.01 | 0.01 | 0.01 |
| | DL-sodium malate | — | — | — | — | 1.00 | — | — | 1.00 |
| | Methyl parahydroxybenzoate | 0.01 | 0.01 | 0.01 | 0.01 | 0.01 | 0.01 | 0.01 | 0.01 |
| | Propyl parahydroxybenzoate | 0.01 | 0.01 | 0.01 | 0.01 | 0.01 | 0.01 | 0.01 | 0.01 |
| | Phenoxyethanol | 0.03 | 0.03 | 0.03 | 0.03 | 0.03 | 0.03 | 0.03 | — |
| | Fucus vesiculosus extract | 0.01 | 0.01 | 0.01 | 0.01 | 0.01 | 0.01 | 0.01 | — |
| | Hydrolyzed silk liquid | 0.01 | 0.01 | 0.01 | 0.01 | 0.01 | 0.01 | 0.01 | 0.01 |
| | Royal jelly extract | 0.01 | 0.01 | 0.01 | 0.01 | 0.01 | 0.01 | 0.01 | 0.01 |
| | Soy protein hydrolysate | — | — | — | — | — | — | — | 0.03 |
| | Licorice extract | 0.07 | 0.07 | 0.07 | 0.07 | 0.07 | 0.07 | 0.07 | 0.07 |
| | Purified water | 72.53 | 71.89 | 73.19 | 73.74 | 72.74 | 72.60 | 71.67 | 74.39 |
| | Total | 100.00 | 100.00 | 100.00 | 100.00 | 100.00 | 100.00 | 100.00 | 100.00 |
| | Viscosity of the first agent | 6.600 | 6.200 | 5.600 | 5.300 | 7.100 | 7.430 | 4.200 | 4.200 |
| | pH of the first agent | 10.6 | 10.6 | 10.4 | 10.4 | 10.3 | 10.3 | 10.3 | 10.7 |
| | | Exam- | Exam- | Exam- | Exam- | Exam- | Exam- | Exam- | Exam- |
| Ingredient name (Second agent) | | ple 42 | ple 43 | ple 44 | ple 45 | ple 46 | ple 47 | ple 48 | ple 49 |
| (A) | Polyoxyethylene (10) cetyl ether | 1.00 | 1.00 | 1.00 | 1.00 | 1.00 | 1.00 | 1.00 | — |
| (A) | Polyoxyethylene (20) cetyl ether | 1.60 | 1.60 | 1.60 | 1.60 | 1.60 | 1.60 | 1.60 | — |
| (A) | Polyoxyethylene (20) stearyl ether | — | — | — | — | — | — | — | 0.26 |
| (A) | Polyoxyethylene (30) stearyl ether | — | — | — | — | — | — | — | 0.20 |
| (B) | Cetanol | 3.70 | 3.70 | 3.70 | 3.70 | 3.70 | 3.70 | 3.70 | 0.75 |
| (B) | Stearyl alcohol | 1.20 | 1.20 | 1.20 | 1.20 | 1.20 | 1.20 | 1.20 | — |
| (C) | Liquid paraffin | 2.00 | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 | 5.81 |
| | Stearyltrimethylammonium chloride | — | — | — | — | — | — | — | 0.24 |
| | Ethanol | — | — | — | — | — | — | — | 0.08 |
| | Diethylene glycol monoethyl ether | 0.02 | 0.02 | 0.02 | 0.02 | 0.02 | 0.02 | 0.02 | — |
| | Polyethylene glycol 400 | 1.50 | 1.50 | 1.50 | 1.50 | 1.50 | 1.50 | 1.50 | — |
| | 1-hydroxyethane-1,1-diphosphonic | 0.14 | 0.14 | 0.14 | 0.14 | 0.14 | 0.14 | 0.14 | 0.12 |
| | acid solution | | | | | | | | |
| | dl-α-Tocopherol | 0.01 | 0.01 | 0.01 | 0.01 | 0.01 | 0.01 | 0.01 | 0.01 |
| | Potassium hydroxide | 0.05 | 0.05 | 0.05 | 0.05 | 0.05 | 0.05 | 0.05 | 0.05 |
| | Phenoxyethanol | 0.04 | 0.04 | 0.04 | 0.04 | 0.04 | 0.04 | 0.04 | — |
| | Purified water | 82.93 | 82.93 | 82.93 | 82.93 | 82.93 | 82.93 | 82.93 | 190.48 |
| | Total | 100.00 | 100.00 | 100.00 | 100.00 | 100.00 | 100.00 | 100.00 | 100.00 |
| | Mixing ratio of the first agent | 1:1 | 1:1 | 1:1 | 1:1 | 1:1 | 1:1 | 1:1 | 1:2 |
| | and the second agent | | | | | | | | |
| | Viscosity of the second agent | 2.300 | 2.300 | 2.300 | 2.300 | 2.300 | 2.300 | 2.300 | 750 |
| | pH of the second agent | 2.6 | 2.6 | 2.6 | 2.6 | 2.6 | 2.6 | 2.6 | 2.7 |
| | Viscosity at the time of mixing | 4.500 | 4.100 | 3.800 | 3.700 | 5.100 | 5.200 | 3.100 | 1.800 |
| | pH at the time of mixing | 10.4 | 10.4 | 10.1 | 10.1 | 9.9 | 9.9 | 9.9 | 10.0 |

The present invention relates to a hair dye composition, according to which, a hair dye composition, in which particularly the first agent and second agent have good preservation stability, and the mixability of the first agent and second agent is good, can be obtained.

What is claimed is:

1. A hair dye composition comprising a first agent containing an alkaline agent and an oxidative dye and a second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C.
